(19) United States
(12) Patent Application Publication
(10) Pub. No.: US 2008/0140077 A1
(43) Pub. Date: Jun. 12, 2008

(54) Femoral Universal Nail

(76) Inventor: Adel Kebaish, Vienna, VA (US)

Correspondence Address: Adam Albrett, 9905 Commonwealth Blvd, Fairfax, VA, 22032-2408, US

(21) Appl. No.: 11/608,818

(22) Filed: Dec. 9, 2006

(51) Int. Cl. A61B 17/58

(52) U.S. Cl. 606/64; 606/62; 606/65

(57) ABSTRACT

An intramedullary nail for insertion in the intramedullary canal of a long bone. The nail includes a nail body having a leading end, a trailing end, and a proximal diametral axis. The trailing end has an axial bore and an intersecting transverse slot. The transverse slot is adapted to pass a sleeve for a lag screw. The sleeve has a sleeve bore axis, and the transverse slot is adapted to provide alternative angles for the sleeve bore axis with respect to the proximal diametral axis. The nail includes a sleeve lock screw. The axial bore includes threads that are adapted to engage complementary threads of the sleeve lock screw. The axial bore is adapted to pass therethrough the sleeve lock screw to make contact with an upper surface of the sleeve when the sleeve is disposed within the transverse slot. A lower surface of the transverse slot and the sleeve lock screw restrict axial movement of the sleeve within the transverse slot and away from the leading end.

### BACKGROUND INFORMATION

Nowadays orthopedic trauma surgeons are faced with increasingly complex injuries to the muscular skeletal system, mainly the femur and the hip joint, due to increasingly high-energy trauma. The high-energy trauma is often caused by construction injuries and automobile related crashes.

The femur is the largest bone in the body. Fractures of the femur can be one, or a combination of the following: femoral neck fractures, complex hip fractures, subtrochanteric fractures, and femoral shaft fractures, including segmental fractures. Other fractures of some complexity often involve the lower part of the femur, otherwise known as the supracondylar fracture. Most often a combination of these fractures can exist in the same femur of the same multiple trauma patient. The treatment can be challenging and demanding.

Over the years, treatment of femoral fractures has evolved due to progress and the development of orthopedic device technology. However, a universal and versatile device for femoral fracture fixation has not been developed. Although many femoral fixation devices have been invented and used over the years, none has been versatile enough to address the complex fractures, or combination of fractures.

Each of the devices was developed to address a limited scope of fractures and most of them inherently have a difficulty addressing the biomechanical instability of complex fracture combinations and especially so in osteoporotic bone. In addition, each of these devices is inherently susceptible to failure if used beyond the limited scope of the fractures it was developed to treat. Accordingly, complex fractures often required the use of a combination of devices on the same patient. This often compromises the safety and the stability of a device, increases operative time and blood loss, and may compromise immediate fracture stability. Furthermore, the need for numerous devices often causes problems in determining how much of any particular device to stock.

Historically, the most common hip fractures, (including femoral neck fracture, intertrochanteric fracture, subtrochanteric fracture) will be treated by a fixed angle hip screw-plate or a fixed angle blade-plate. A fixed angle device has an inherent problem of not allowing compression and sliding at the fracture site, which often leads to many failures These devices have been abandoned except for a very limited set of fractures.

The dynamic hip screw was later developed, and it solved some of the problems with the fixed angle devices. The dynamic hip screw has been found useful for treating stable hip fractures, but was never found to be suitable or indicated for the unstable hip fractures, especially the subtrochanteric fractures and reversed angle intertrochanteric fractures. Moreover dynamic hip screws/plates do not address a combination of hip fracture and femoral shaft fracture, and certainly do not address complex, unstable femoral shaft fractures. In addition, the foregoing two devices require an extensive surgical approach which may lead to increased bleeding and devitalization of bone fragment and soft tissue.

The intramedullary rod (IM nail) is commonly used in treating femoral shaft fractures. The main indication for its use is primarily femoral shaft fractures. The IM nail will not address a subtrochanteric fracture or a combination of femoral neck/intertrochanteric fractures and even more so for supracondylar femur fractures, or a combination of the foregoing fractures.

Over the years some adaptations to the IM nails (e.g., gamma nail, recon nails) were made to enable fixation to the proximal femur. Fixation is usually done by adding one or two screw fixations to the femoral neck and head. However, this does not solve the problem of complex and combination fractures. The proximal screw fixation acted only as anchoring screws without allowing any sort of compression at the proximal fracture site, whether femoral neck, hip, or subtrochanteric.

It is always difficult to place the proximal screws in the optimum location since they had to be introduced through a hole or two holes in the nail that provided limited flexibility with regards to angle of placement of the proximal screw(s) relative to the fracture geometry. This often leads to a deforming force across the proximal fracture site, more so if there is a combination of fractures in the same femur, proximal (femoral neck) or distal (shaft).

No IM nail is available to address the complex intercondylar and supracondylar distal femoral fractures. Those usually are treated by a variety of plates—locking and non-locking—which often result in significant soft tissue stripping and devitalization of bone fragments.

FIG. 1 illustrates a common intramedullary nail used to treat femoral shaft fractures. Intramedullary nail 10 is suitable for treating fractures such as those that are located in the shaft of the femur. Nail 10 cannot be used to address a distal femur fracture or a subtrochanteric/intertrochanteric fracture, femoral neck fracture or a combination of the foregoing. If after surgery and recovery a fracture were to appear in the neck of the femur and surgical treatment were pursued, intramedullary nail 10 would most probably be removed and a gamma nail or recon nail inserted in its place.

FIG. 3 illustrates a gamma nail and a screw that can be inserted in a femoral head. The axis of screw 31 is at an angle W with respect to the proximal diametral axis (dashed line in FIG. 3) of gamma nail 30. Gamma nails come with screw bores that have a fixed angle W, which may not be suitable for every fracture geometry and variable proximal hip anatomy. For example, if a 130° gamma nail and screw are inserted and it appears that the screw cannot be placed in the most desirable and stable bony location, the gamma nail and screw would have to be removed and another gamma nail with a different angle would have to be inserted in its place. Currently, gamma nails are available at two angles of insertion which means that for nails of every size two nails each with a different angle of insertion must be stocked. Even though inventory is doubled at most two angles of insertion are available for each nail size. Due to the limited angles of insertion the fracture often is forced into valgus or varus mal-alignment. Gamma nails do not provide for compression across the fracture site.

Mal-alignments could lead to failure of fixation, non-union, mal-union, or delayed union which may adversely affect the ultimate result of the surgery.

FIG. 2a illustrates a gamma nail and screw with mal-alignment of the varus type. Mal-alignment often happens because the bore in the gamma nail through which screw 22 passes limits the angle at which screw 22 can enter the head. Screw 22 has caused varus mal-alignment between the femoral head and the neck of the femur as indicated by exposed regions 26.

A non-compressive screw used with a gamma nail can be locked or unlocked. If it is locked it will control deformity due to rotation of the screw, but will not allow sliding. If it is unlocked, it will allow sliding but will not control rotation of the screw. The non-compressive screw acts as an anchor rather than a lag screw that provides compression across the fracture.

FIG. 2b illustrates a gamma nail and screw with mal-alignment of the valgus type. Screw 22 has caused valgus mal-alignment as indicated by exposed regions 27. It is not possible to predict definitively what type of mal-alignment, if any, will occur.

FIG. 4a is a longitudinal cross section view of a dynamic hip screw system placed in a bone. System 44 includes a plate and barrel combination 46, lag screw 47, and compression screw 49. FIG. 4d is a front view of the plate and barrel combination of FIG. 4a. FIG. 4e is a view, in longitudinal cross section, of the plate and barrel combination of FIG. 4a.

Combination 46 includes holes 21 for driving screws 48 into the bone in order to attach combination 46 to the bone. Screw 49 has outer threads that can be driven into inner threads (not shown) in the trailing edge of screw 47. Since screw 49 pushes against a counterbore 11 in combination 46 a compressive force is applied across the fracture. A dynamic hip screw does not treat a combination of intertrochanter fractures and fractures in the femoral shaft, including the subtrochanter region, due to the excessive forces across these fractures. Using a dynamic hip screw in a first surgical operation complicates matters for a subsequent surgical operation which is supposed to treat new fractures in the shaft, subtrochanter, and intertrochanter regions. Furthermore, combination 46 and screws 48 act as a load bearer which is less preferable to a system that provides load sharing. System 44 suffers from the same shortcomings of the fixed-angle plate-blade, including mal-alignments, limited angles of insertion, and lack of load sharing. Lack of load sharing may lead to delayed range of motion and weight bearing as well as frequent hardware failure, which may lead to mal-union, or non-union. Dynamic hip screw will require an extensive surgical approach which may lead to soft tissue and bone devitalization.

FIG. 4b is a longitudinal cross section view of a plate used to treat fractures in the distal femur. FIG. 4c is a front view of the plate of FIG. 4b. Plate 40 includes holes 41 through which screws 42 pass. These plates can be locking and none locking. Depending on the number fractures and their location along the shaft, plate 40 can be quite large and will require an incision that is at least equal to the length of plate being used which extends operative time and blood loss. Plate 40 is affixed to the bone using multiple screws 42. Plate 40 also acts as a load bearer which is less preferable to intramedullary devices which act as load sharers and allow an axially compressive force be applied to reduce the gaps along fracture lines. Instead of requiring hospitals to stock plates of various sizes, it would be preferable to have a device that can be used in the context illustrated in FIG. 4b in addition to the other contexts described above and that would act as a load sharer.

Prior art solutions suffer from many shortcomings, some of which are identified herein. As described above, current devices are limited in the number of cases they can treat causing problems with respect to stocking the appropriate types and number of devices. The limited angles of insertion provided by current devices promote mal-alignment. Given the shortcomings of the prior art, it is desirable to provide solutions that allow surgeons to provide treatment that overcomes deficiencies of the prior art.

### SUMMARY OF THE INVENTION

In an embodiment, an intramedullary nail for insertion in the intramedullary canal of a long bone includes a nail body having a leading end and a trailing end and a proximal diametral axis. The trailing end has an axial bore and an intersecting transverse slot. The transverse slot is adapted to pass a sleeve for a lag screw. The sleeve has a sleeve bore axis, and the transverse slot is adapted to provide alternative angles for the sleeve bore axis with respect to the proximal diametral axis. The nail includes a sleeve lock screw. The axial bore includes threads that are adapted to engage complementary threads of the sleeve lock screw. The axial bore is adapted to pass therethrough the sleeve lock screw to make contact with an upper surface of the sleeve when the sleeve is disposed within the transverse slot. A lower surface of the transverse slot and the sleeve lock screw restrict axial movement of the sleeve within the transverse slot and away from the leading end.

### BRIEF DESCRIPTION OF THE DRAWINGS

The present invention is illustrated by way of example, and not limitation, in the figures of the accompanying drawings in which like references denote similar elements, and in which:

FIG. 1 is a view, in a longitudinal cross section, of a common intramedullary nail placed in the intramedullary canal of a fractured bone;

FIG. 2a is a view, in a longitudinal cross section, of a gamma nail placed in the intramedullary canal of a fractured bone with a screw resulting in mal-alignment of the varus type;

FIG. 2b is a view, in a longitudinal cross section, of a gamma nail placed in the intramedullary canal of a fractured bone with a screw resulting in mal-alignment of the valgus type;

FIG. 3 is a longitudinal view of a gamma nail and screw that can be inserted in a femoral head;

FIG. 4a is a view, in longitudinal cross section, of a dynamic hip screw attached to a bone with a lag screw placed in the intratrochanter, femoral head, and femoral neck regions of the bone;

FIG. 4b is a view, in a longitudinal cross section, of a plate with screws placed in the distal femur;

FIG. 4c is a front view of the plate of FIG. 4b;

FIG. 4d is a front view of the plate and barrel combination of FIG. 4a;

FIG. 4e is a view, in longitudinal cross section, of the plate and barrel combination of FIG. 4a;

FIG. 5a is a longitudinal view of an intramedullary nail system according to an embodiment of the present invention in an exploded state;

FIG. 5b is a front view of the nail body of FIG. 5a according to an embodiment of the present invention;

FIG. 5c is a longitudinal view of a nail body according to an alternative embodiment of the present invention;

FIG. 5d is a front view of the nail body of FIG. 5c according to an alternative embodiment of the present invention;

FIG. 5e is a view, in a longitudinal cross section, of the nail body of FIG. 5c according to an alternative embodiment of the present invention;

FIG. 5f is a longitudinal view of a nail body according to an alternative embodiment of the present invention;

FIG. 5g is a front view of the nail body of FIG. 5f according to an alternative embodiment of the present invention;

FIG. 5h is a view, in a longitudinal cross section, of the nail body of FIG. 5f according to an alternative embodiment of the present invention;

FIG. 6a is a view, in a longitudinal cross section, of a nail body according to an embodiment of the present invention;

FIG. 6b is a view, in a longitudinal cross section, of a nail body according to an embodiment of the present invention with a screw at an angle of 150° with respect to the proximal bore axis of the nail body;

FIG. 6c is a view, in a longitudinal cross section, of a nail body according to an embodiment of the present invention with a screw at an angle of 135° with respect to the proximal bore axis of the nail body;

FIG. 6d is a view, in a longitudinal cross section, of a nail body according to an embodiment of the present invention with a screw at an angle of 125° with respect to the proximal bore axis of the nail body;

FIG. 6e is a view, in a longitudinal cross section, of a nail body according to an embodiment of the present invention with a screw at an angle of 135° with respect to the proximal bore axis of the nail body and where the lower surface of the slot in the nail body is touching the lower surface of a sleeve;

FIG. 6f is a view, in a longitudinal cross section, of a nail body according to an embodiment of the present invention with a screw at an angle of 90° with respect to the proximal bore axis of the nail body;

FIG. 6g is a view, in a longitudinal cross section, of a nail body according to an embodiment of the present invention with two screws placed through a slot;

FIG. 7a is a view, in longitudinal cross section, of an intramedullary nail system according to an embodiment of the present invention placed in the intramedullary canal of a bone;

FIG. 7b is an enlarged view of the proximal portion of the intramedullary nail system of FIG. 7a;

FIG. 7c illustrates a washer according to an embodiment of the present invention;

FIG. 7d illustrates a washer according to an alternative embodiment of the present invention;

FIG. 8 is an isometric view of a sleeve according to an alternative embodiment of the present invention;

FIG. 9 is a top of view of FIG. 8;

FIG. 10 is a cross section view of FIG. 8;

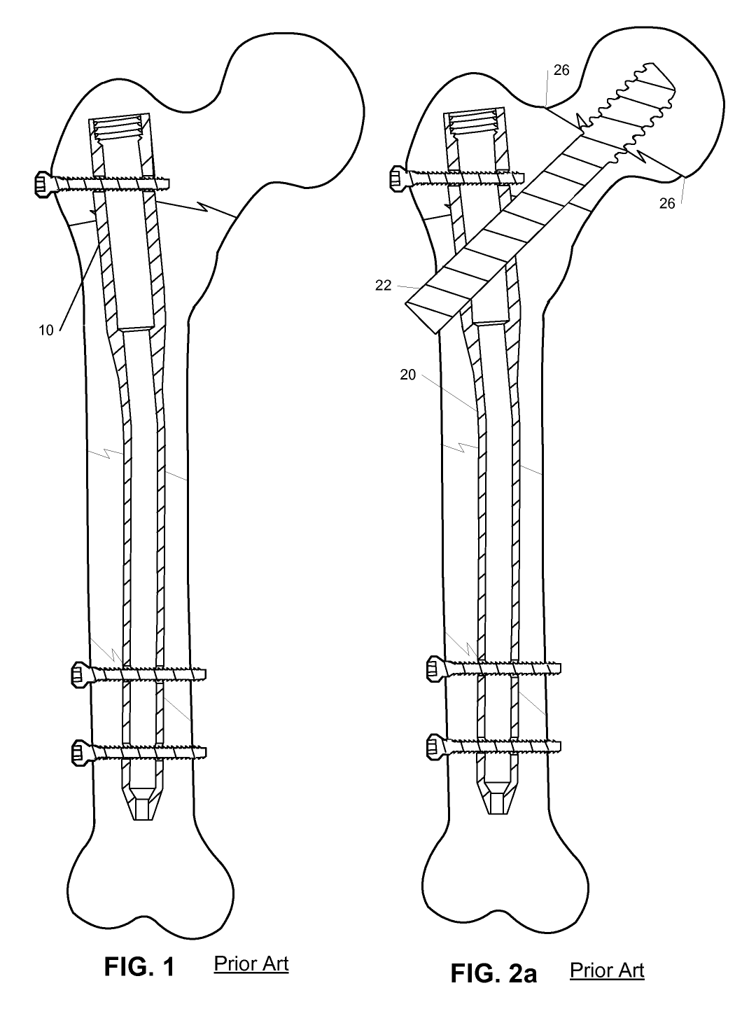

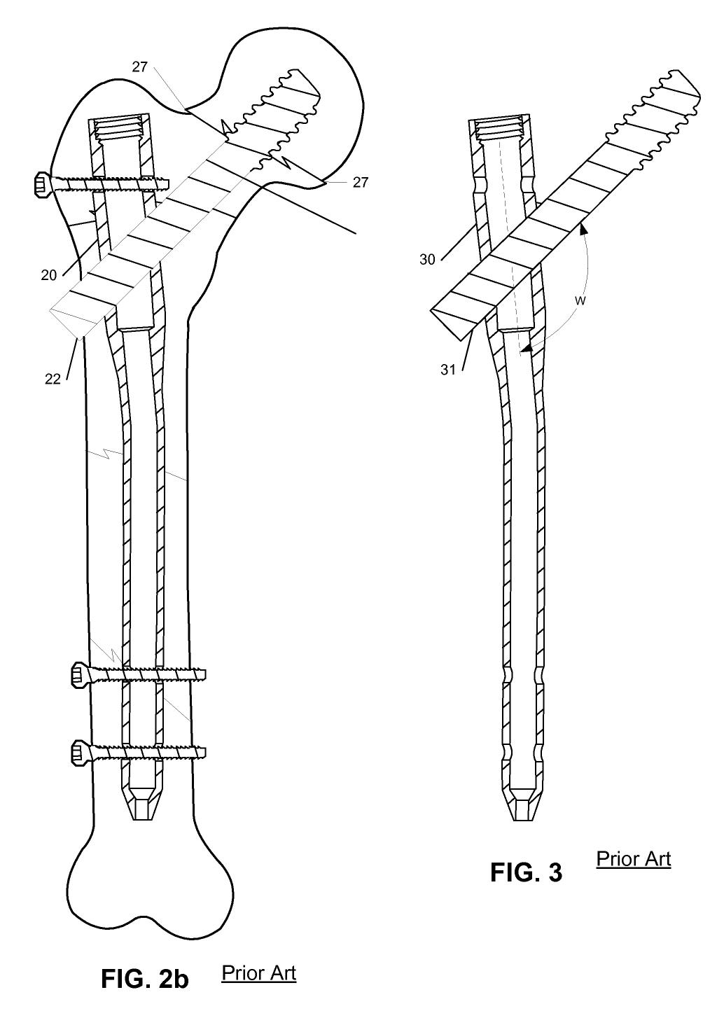

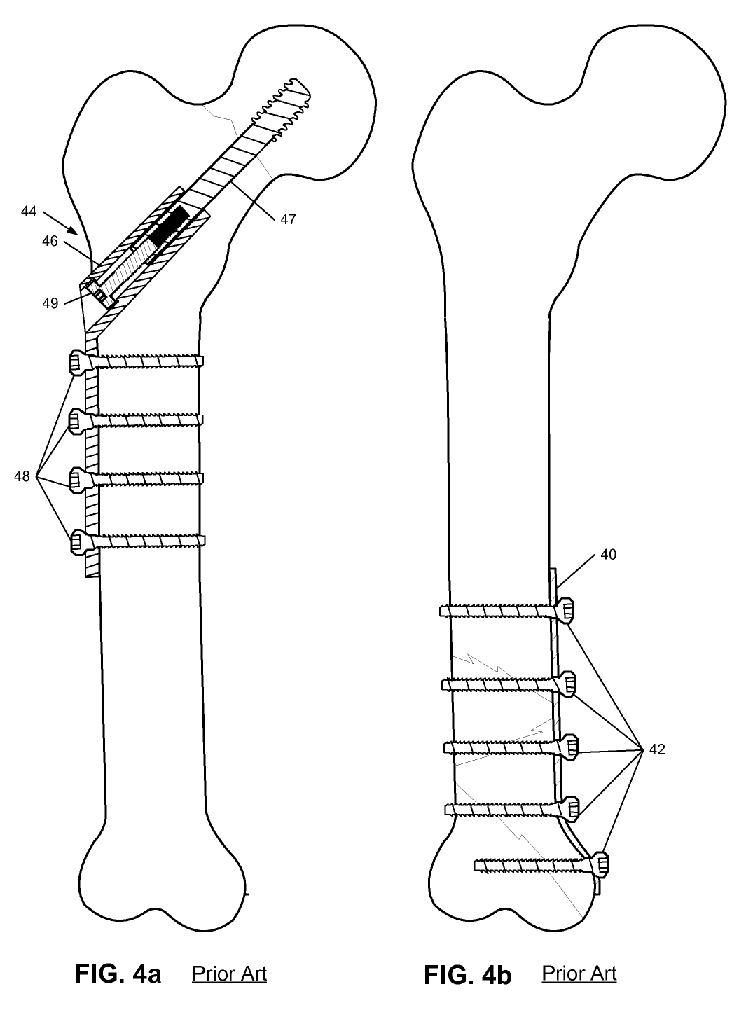

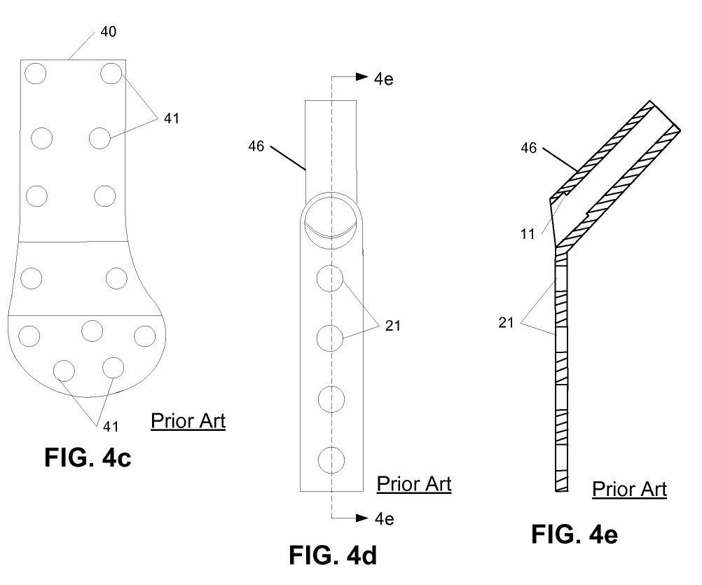

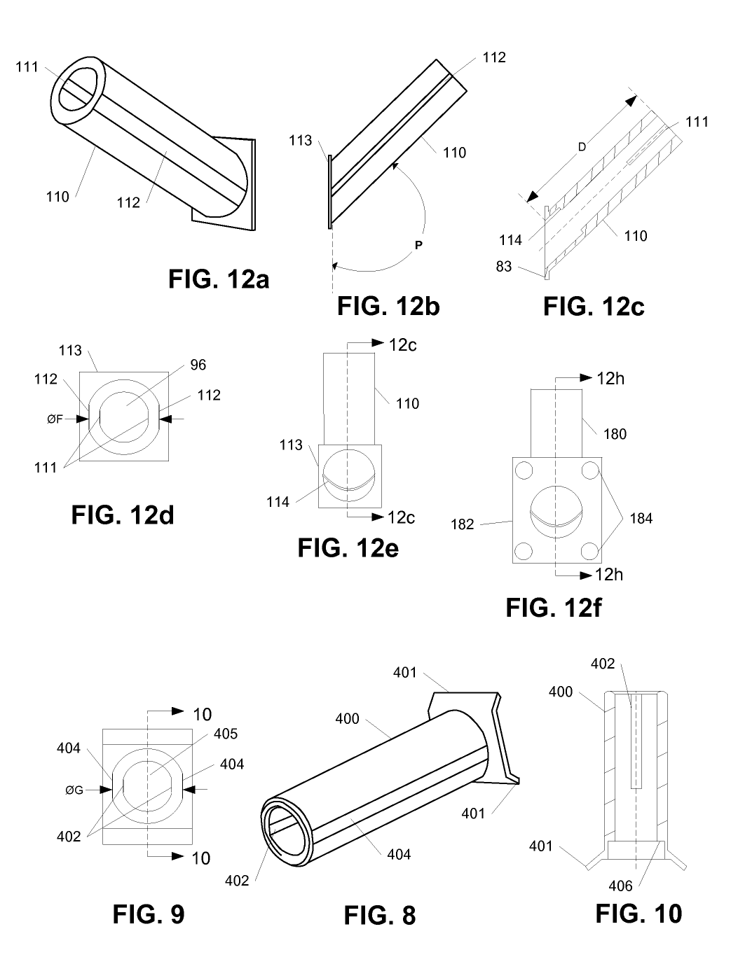

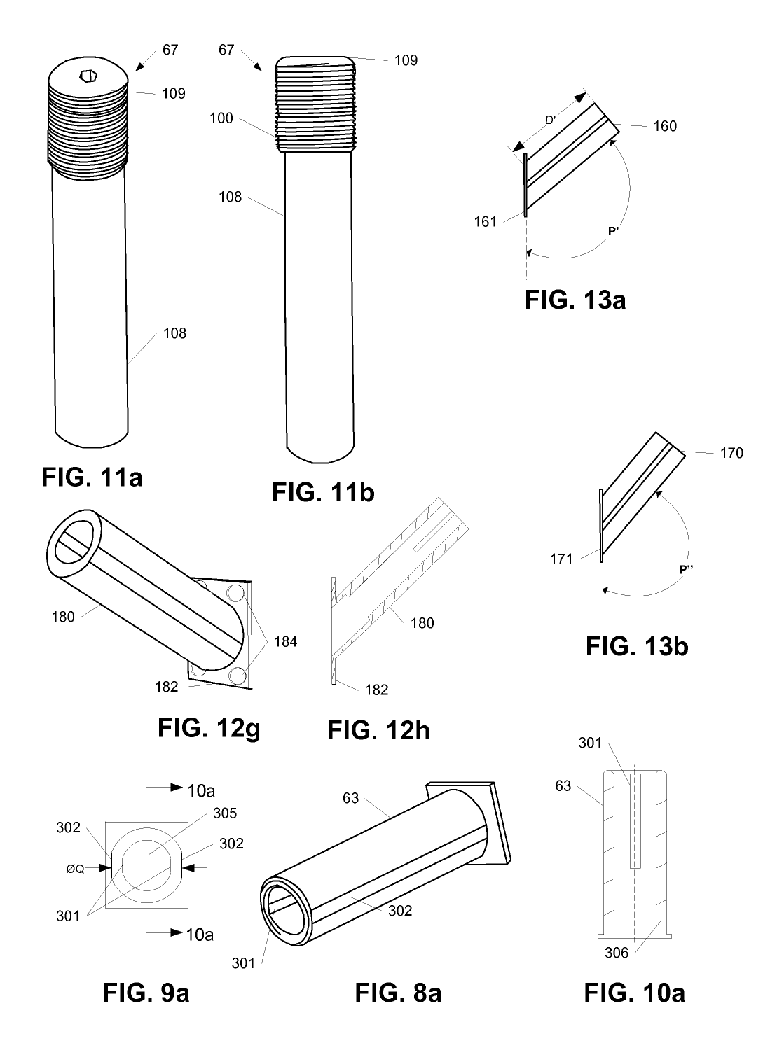

FIG. 8a is an isometric view of a sleeve according to an alternative embodiment of the present invention;

FIG. 9a is a top of view of FIG. 8a;

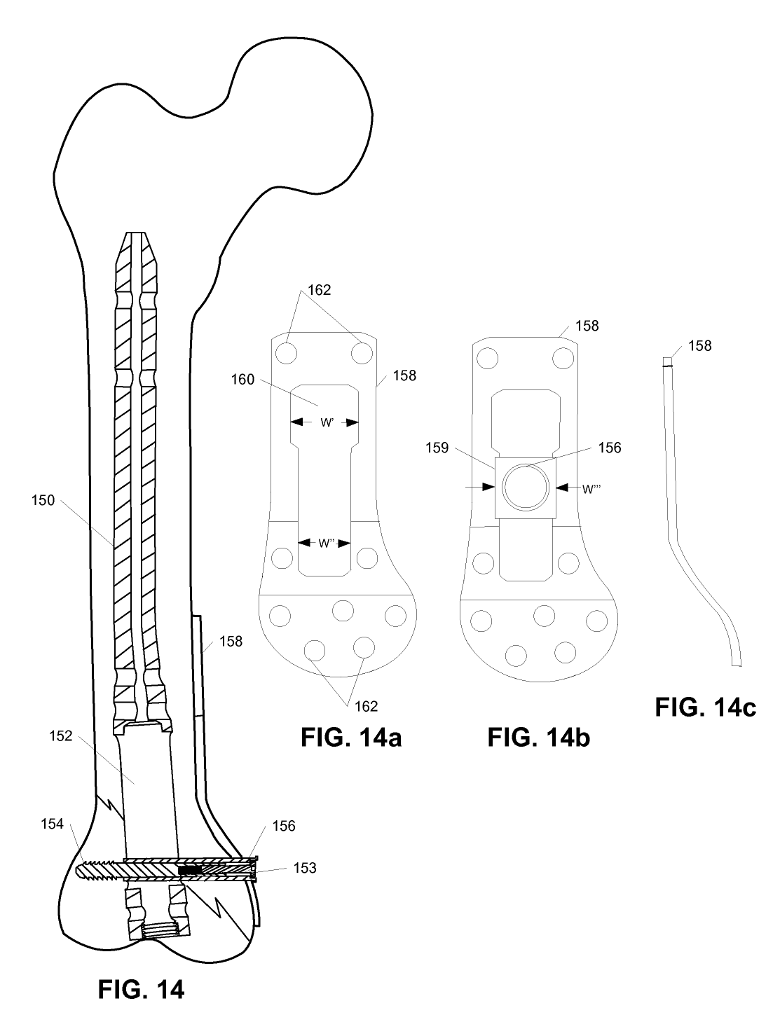

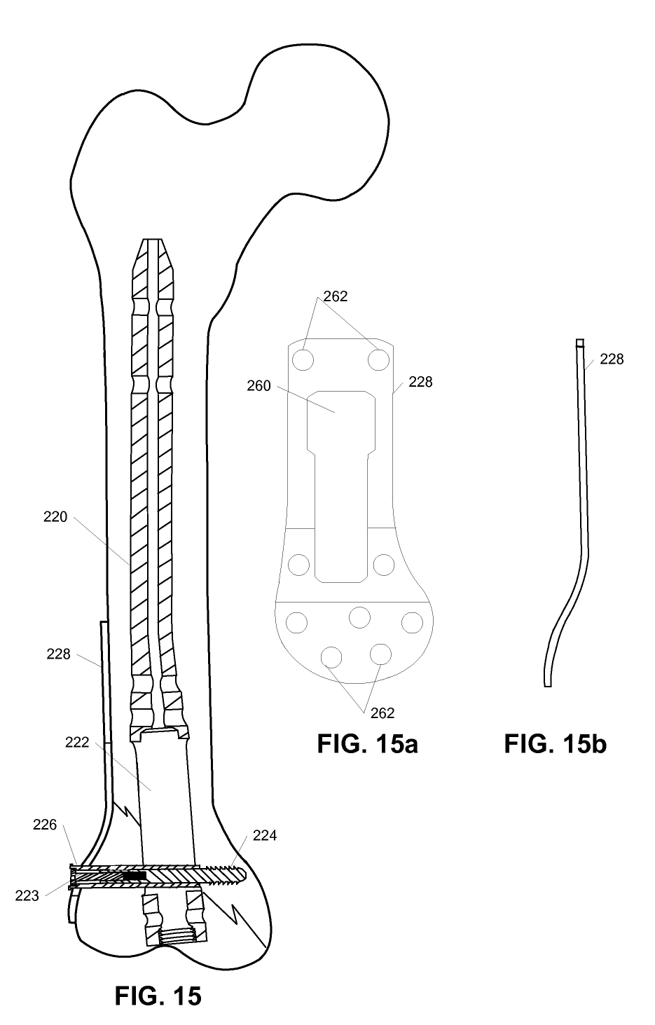

FIG. 10a is a cross section view of FIG. 8a;

FIG. Ha is an isometric view of a sleeve lock according to an embodiment of the present invention;

FIG. 11b is a front view of FIG. 11a;

FIG. 12a is an isometric view of a sleeve according to an alternative embodiment of the present invention;

FIG. 12b illustrates a side view of the sleeve of FIG. 12a according to an alternative embodiment of the present invention;

FIG. 12c is a cross section view of the sleeve of FIG. 12a;

FIG. 12d is a view from the front of the sleeve of FIG. 12a and looking down the sleeve bore.

FIG. 12e is a front view of the sleeve of FIG. 12a according to an alternative embodiment of the present invention.

FIG. 12f is a front view of a sleeve according to an alternative embodiment of the present invention;

FIG. 12g is an isometric view of the sleeve of FIG. 12f;

FIG. 12h is a cross section view of the sleeve of FIGS. 12f, 12g;

FIG. 12i is a cross section view of a sleeve according to an alternative embodiment of the present invention;

FIG. 12j is another cross section view of the sleeve FIG. 12i with a compression screw and an end cap restraining the compression screw;

FIG. 12k is an isometric view of an end cap for a sleeve according to an embodiment of the present invention;

FIG. 12l is a front view of the end cap of FIG. 12k.

FIG. 13a is a side view of a sleeve according to yet another alternative embodiment;

FIG. 13b is a side view of a sleeve according to yet another alternative embodiment;

FIG. 14 is a view, longitudinal cross section, of an intramedullary nail system according to an alternative embodiment of the present invention placed in the intramedullary canal of a bone in a retrograde fashion with a lock plate disposed medially;

FIG. 14a is a front view of the lock plate in FIG. 14 according to an alternative embodiment of the present invention;

FIG. 14b is a front view of the lock plate in FIG. 14 and the sleeve of FIG. 14 as placed in the opening of the washer according to an alternative embodiment of the present invention;

FIG. 14c is a side view of the lock plate in FIGS. 14, 14a, 14b;

FIG. 15 is a view, in longitudinal cross section, of an intramedullary nail system according to an alternative embodiment of the present invention placed in the intramedullary canal of a bone in a retrograde fashion with a lock plate disposed laterally;

FIG. 15a is a front view of the lock plate in FIG. 15 according to an alternative embodiment of the present invention; and

FIG. 15b is a side view of the lock plate in FIGS. 15, 15a.

### DETAILED DESCRIPTION

According to the present invention, devices for treating fractures are described. Devices each of which allows a screw to be inserted across a fracture at one of several angles are also described. Furthermore, devices each of which allows a compressive force to be applied across a fracture at one of several angles are also described. In the following description, for purposes of explanation, numerous specific details are set forth in order to provide a thorough understanding of embodiments according to the present invention. It will be evident, however, to one of ordinary skill in the art that the present invention may be practiced in a variety contexts including treatment of femoral fractures without these specific details. In other instances, well-known operations, steps, functions and elements are not shown in order to avoid obscuring the description.

Parts of the description will be presented using terminology commonly employed by those skilled in the art to convey the substance of their work to others skilled in the art, such as cortical screw, intramedullary nail, axial bore, and so forth. Various operations will be described as multiple discrete steps performed in turn in a manner that is most helpful in understanding the embodiments according to the present invention. However, the order of description should not be construed as to imply that these operations are necessarily performed in the order that they are presented, or even order dependent. Lastly, repeated usage of the phrases “in an embodiment,” “an alternative embodiment,” or an “alternate embodiment” does not necessarily refer to the same embodiment, although it may.

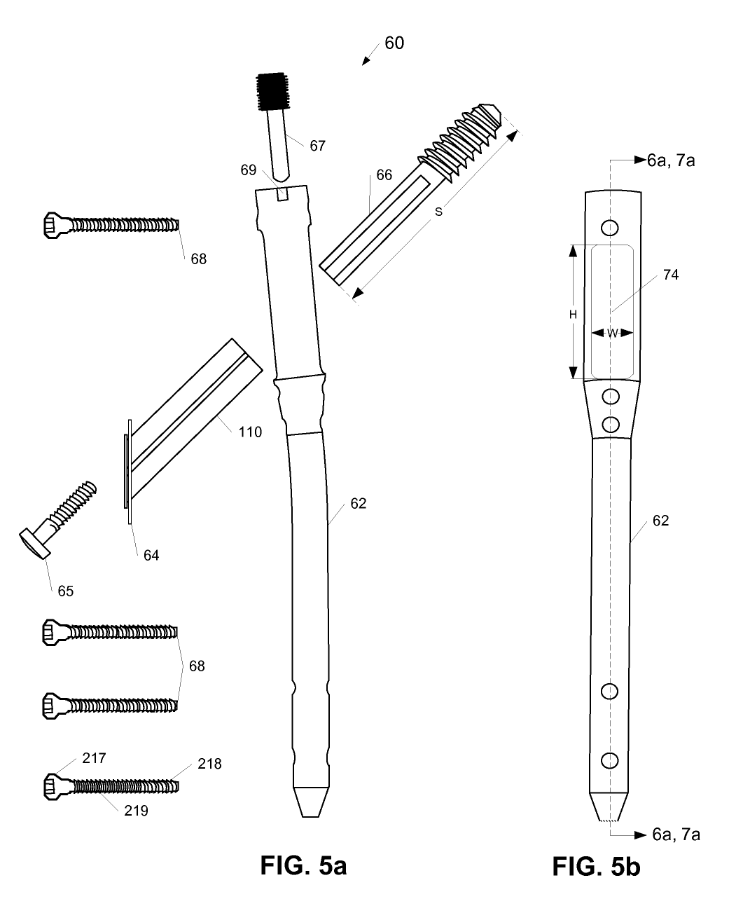

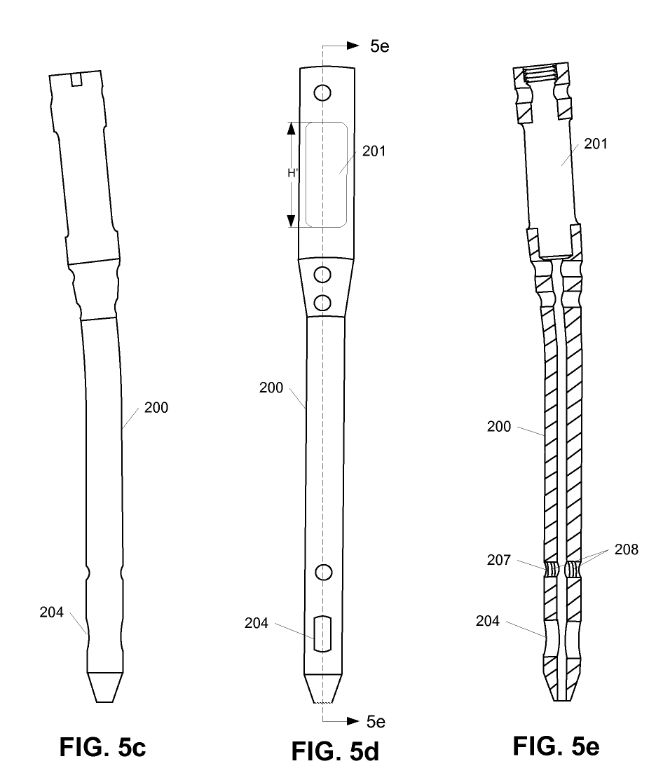

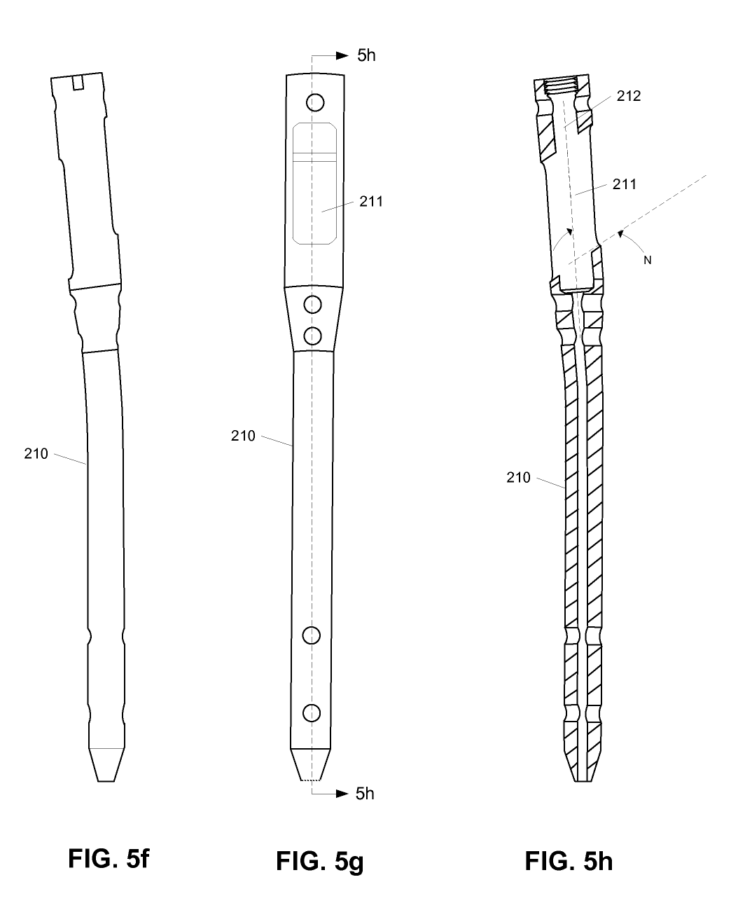

FIG. 5a is a longitudinal view of an intramedullary nail system according to an embodiment of the present invention in an exploded state. Nail system 60 is constructed of implantable grade stainless steel alloys in an embodiment, but could also be constructed of implantable grade titanium, titanium alloys or polymeric materials such as nylon, carbon fibers and thermoplastics, as well. System 60 includes nail body 62, sleeve 110, washer 64, compression screw 65, lag screw 66, sleeve lock screw 67 and cortical screws 68.

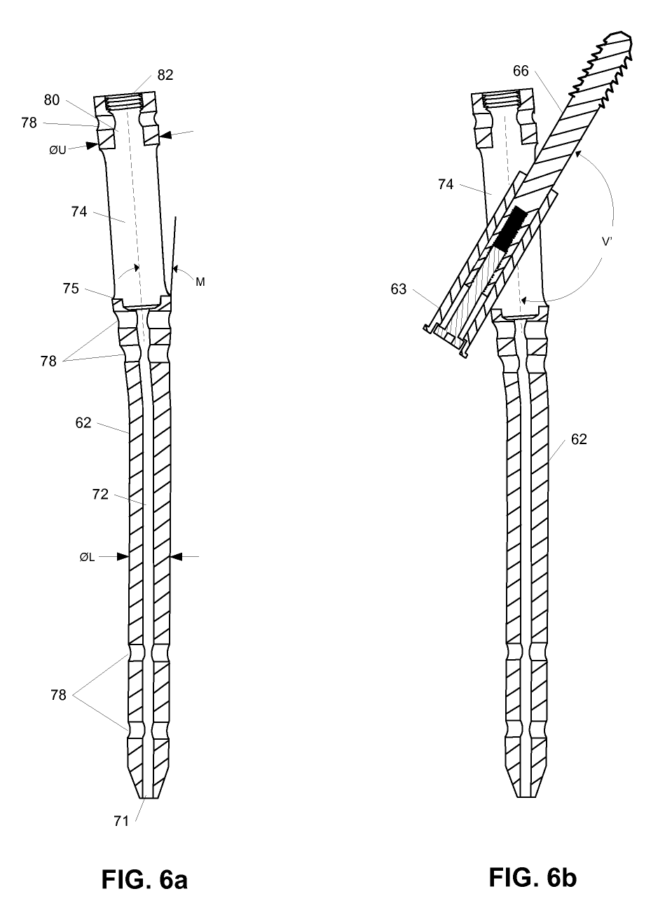

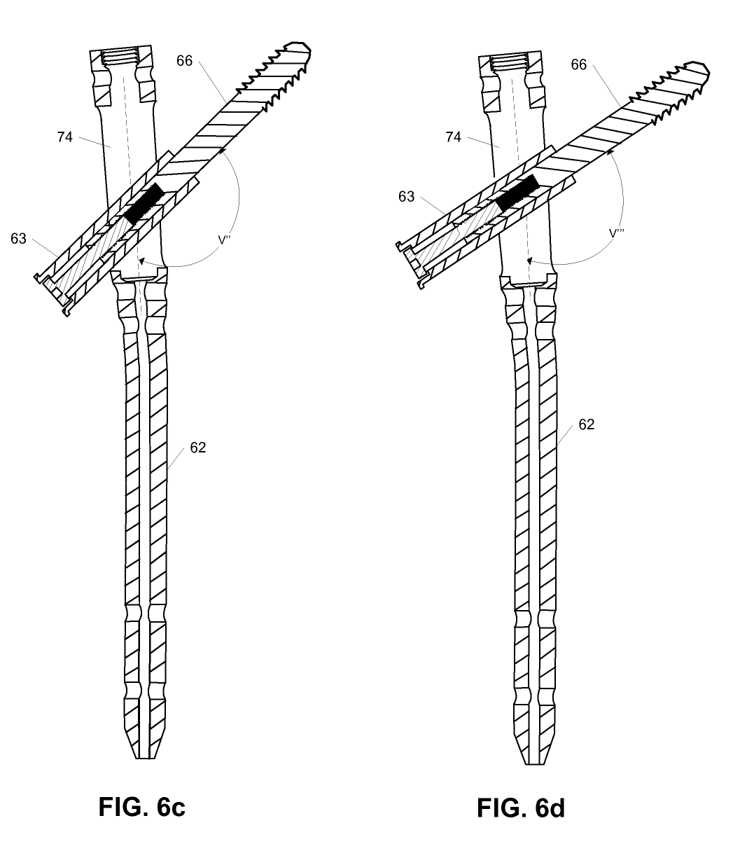

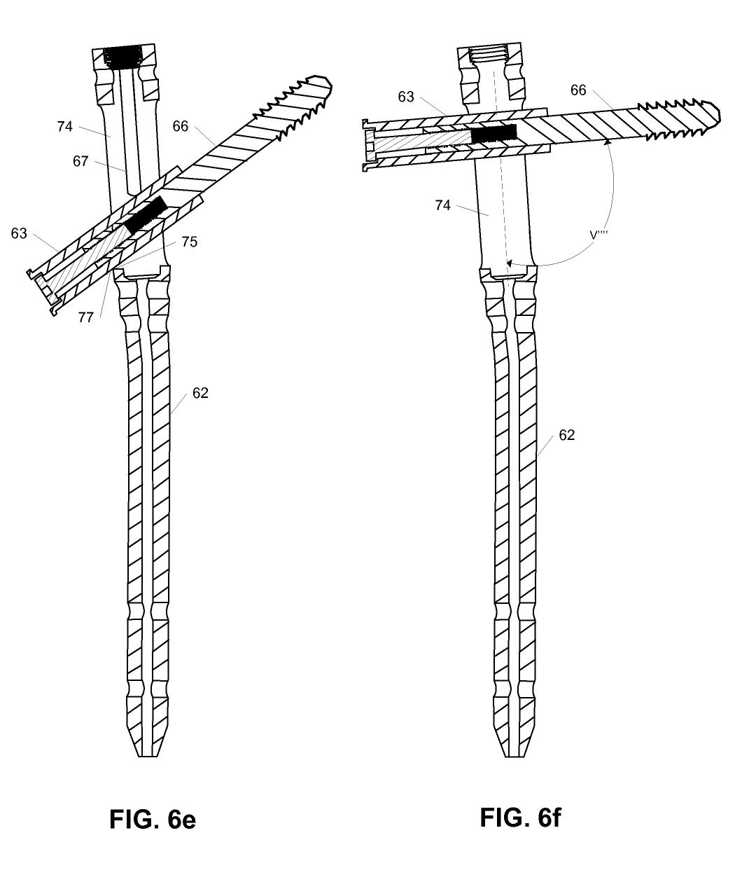

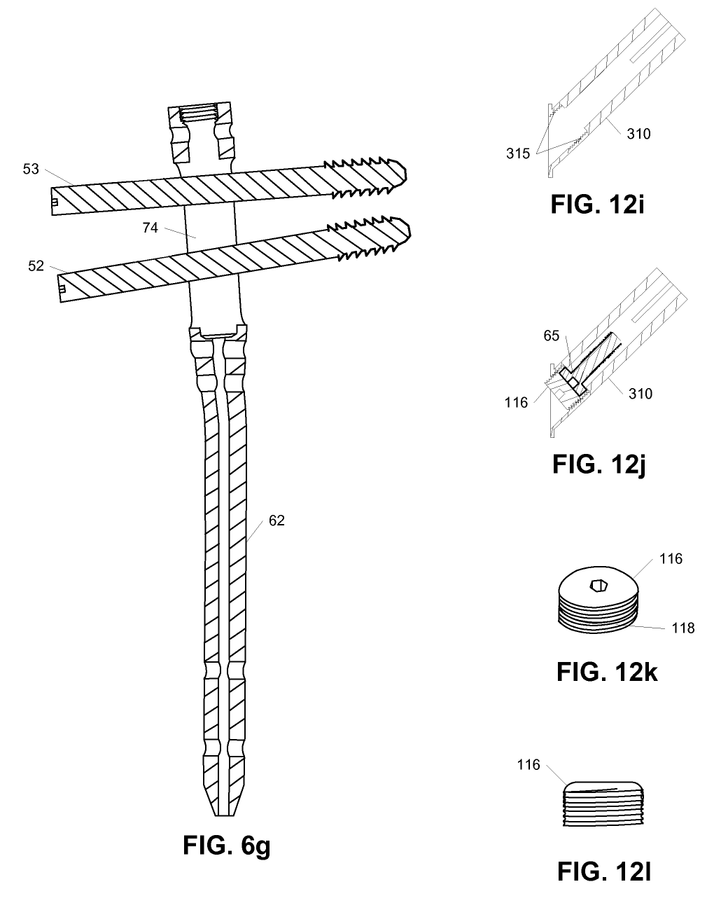

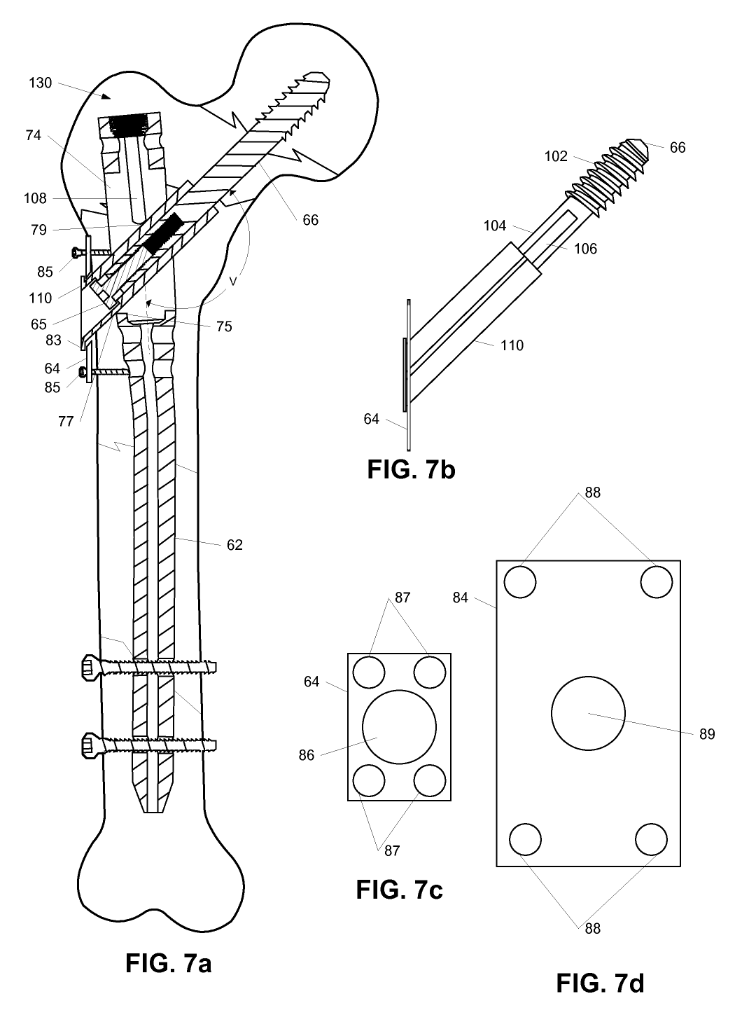

FIG. 6a is a view, in a longitudinal cross section, of a nail body according to an embodiment of the present invention. Nail body 62 can be inserted either in an antegrade fashion or a retrograde fashion into the intramedullary canal of the femur. In an embodiment, the proximal outside diameter U of the body is greater than the distal outside diameter L due to narrowing of the canal and to allow slot 74 to be large enough to pass threads 102 (FIG. 7b) of lag screw 66 and allow sleeve 110 to slide into slot 74 and be positioned at various angles V in relation to the proximal bore axis (dashed line in slot 74) (FIGS. 5h, 6a-6d, 6f, 7a). FIG. 7a is a view, in longitudinal cross section, of an intramedullary nail system according to an embodiment of the present invention placed in the intramedullary canal of a bone. FIG. 7b is an enlarged view of the proximal portion of the intramedullary nail system of FIG. 7a.

In an alternative embodiment, nail body 62 is curved in a manner which allows for trochanteric insertion as well as adaptation to the anatomy of the femur. In the case where nail body 62 is inserted in an antegrade fashion, slot 74 allows lag screw 66 to be inserted into the head of the femur at a wide range of angles. In the case where nail body 62 is inserted in a retrograde fashion, slot 74 allows lag screw 66 to be inserted into the distal femur or femoral condyles at a wide range of angles.

FIG. 8a is an isometric view of a sleeve according to an alternative embodiment of the present invention. FIG. 9a is a top of view of FIG. 8a. FIG. 10a is a cross section view of FIG. 8a. Body 104 of lag screw 66 has two flats 106 180° apart (FIG. 7b) which interface with bore 305 and flats 301 (FIGS. 8a, 9a, 10a) of sleeve 63 in such a way as to allow axial translation or slide of the lag screw while preventing rotation relative to sleeve 63. This sliding prevents penetration of the femoral head by the proximal end of the lag screw as the fracture compresses from patient load bearing.

FIG. 6b is a view, in a longitudinal cross section, of a nail body according to an embodiment of the present invention with a screw at an angle of 150° with respect to the proximal bore axis of the nail body. The axes of the bores of lag screw 66 and sleeve 63 are both at an angle V′ with respect to the proximal bore axis (dashed line in slot 74) (FIG. 6b). In an embodiment, angle V′ can be varied between 90° and 150° allowing lag screw 66 to be properly positioned within the femoral head. In FIG. 6b, angle V′ is 150°. Providing a surgeon with the flexibility to insert lag screw 66 into the femoral head at wide range of angles is desirable because it allows the surgeon to achieve better compression across a fracture—in an embodiment in which a lag screw is used, reduce the possibility of getting varus or valgus alignment, and improve the prospects of the bone healing properly.

As illustrated in FIGS. 6b, 6c, 6d, 6e, 6f, slot 74 of nail body 62 allows screw 66 to be inserted into the head of the femur at a range of angles extending from 150° to 90°. FIG. 6c is a view, in a longitudinal cross section, of a nail body according to an embodiment of the present invention with a screw at an angle of 135° with respect to the proximal bore axis of the nail body. Angle V″ is 135°. FIG. 6d is a view, in a longitudinal cross section, of a nail body according to an embodiment of the present invention with a screw at an angle of 125° with respect to the proximal bore axis of the nail body. Angle V′″ is 125°.

FIG. 6e is a view, in a longitudinal cross section, of a nail body according to an embodiment of the present invention with a screw at an angle of 135° with respect to the proximal bore axis of the nail body and where the lower surface of the slot in the nail body is touching the lower surface of a sleeve. When sleeve 63 is fixed axially in slot 74 and sleeve 63 is not touching lower surface 75 of slot 74, screw 67, when screwed into threads 82 (FIG. 6a) and axial bore 80 raises, nail body 62 relative to sleeve 63 such that lower surface 75 of slot 74 is touching lower surface 77 of sleeve 63. Alternatively, an insertion/removal instrument (not shown) as described elsewhere may be used to pull on nail body 62 so that surface 75 is raised to touch surface 77.

FIG. 6f is a view, in a longitudinal cross section, of a nail body according to an embodiment of the present invention with a screw at an angle of 90° with respect to the proximal bore axis of the nail body. Angle V″″ is 90°.

FIG. 6g is a view, in a longitudinal cross section, of a nail body according to an embodiment of the present invention with two screws placed through a slot. Slot 74 of nail body 62 has two screws 52, 53 passing through it. Allowing two screws to be independently inserted into the proximal femur is a significant benefit of nail body 62 and the nail bodies of other embodiments of the invention described herein.

FIG. 5b is a front view of the nail body of FIG. 5a according to an embodiment of the present invention. It would be apparent to one of ordinary skill in the art that the range of angles at which a screw can be inserted depends upon the height of the slot and the relative dimensions of the slot and the sleeve. Furthermore, one of ordinary skill in the art would appreciate that the height H of slot 74 (FIG. 5b) is a design factor that can vary depending upon the range of angles V that a given nail system is designed to support, and need not be specified in any detail herein. In an alternative embodiment, slot 74 limits the angle of insertion to values greater than or equal to x° and less than or equal to y°, where x°<y° and x°≧90° and y°≦150°.

While slot 74 is a hole in the shape of a box with height H and width W (FIG. 5b), one of ordinary skill in the art would appreciate that in an alternative embodiment slot 74 is a circular bore with a diameter larger than the outer diameter of sleeve 110, thereby allowing a sleeve and screw 66 to be pointed in more than one angle. One of ordinary skill in the art would appreciate that in such an alternative embodiment the sleeve would be locked in relative rotation by a sleeve lock screw with a shaped surface that engages a shaped surface of the sleeve to prevent relative rotation. One of ordinary skill in the art would appreciate that width W is also a design factor that may depend—in an embodiment—on the strength of the material from which nail body 62 is made and need not be specified in any detail herein.

One of ordinary skill in the art would appreciate that wherever sleeve 110 is referred to in an embodiment, in an alternative embodiment another sleeve described herein is used in the alternative embodiment.

In the case where a first operation is performed to treat fractures in the shaft or subtrochanter region of the femur and a second operation is needed later to treat fractures that appear later in the head or neck of the femur, it is very significant that nail body 62 allows a wide range of angles of insertion. When a second operation is needed to treat fractures in the head of the femur, slot 74 allows lag screw 66 to be inserted into the femoral head at a wide range of angles making it very likely or even certain that the surgeon will be able to insert screw 66 at the appropriate angle. Fixed-angle nails do not provide such a range of choices with respect to angles of insertion which means treatment with fixed-angle nails in a first operation generally results in far less than optimal treatment of femoral head fractures in a second operation or necessitates the removal of the fixed-angle nail from the femur and replacement by another fixed-angle nail that has a different angle of insertion. Often the fixed-angle nail that has a different angle of insertion is also not suitable because the angle of insertion which is preferable is not supported by any fixed-angle nail that is available.

Nail body 62 can be inserted in a retrograde fashion in the femur to treat complex fractures in the lower femoral shaft and the distal femur. Even though nail body 62 is anatomically shaped to the axis of the canal and has a mediolateral bend angle M (FIG. 6a), the nail body 62 can be inserted in a retrograde fashion into the canal as described in greater detail below. Slot 74 allows lag screw 66 or a standard screw (non-lag screw) to be inserted to treat fractures in the distal femur. Using nail body 62, lag screw 66 or a non-lag screw to treat complex fractures is preferable to using plates because they allow for stabilization and reduction of fractures and load sharing rather than just load bearing. Retrograde insertion is described in greater detail below.

The foregoing applications and attributes of nail 60 and other applications and attributes described herein are very significant because they permit a device such as nail 60 to treat a greater variety of combinations of femoral fractures than is possible with current nails. Having a device such as nail 60 treat a greater variety of femoral fractures means that fewer types of devices needed to be stocked and increases the possibility that a device will be available in stock when needed for surgery.

One of ordinary skill in the art would appreciate that wherever lag screw 66 is described herein, in alternative embodiments, a non-lag screw may be used instead. In each case where a non-lag screw is used a sleeve, such as sleeve 110, may be unnecessary.

Having described some of the applications and benefits of nail 60, some of the steps associated with inserting nail body 62 in an antegrade fashion into the femur will be described. Prior to insertion, a guide pin (not shown) is inserted into the femur at the trochanter region and driven towards the distal end of the femur. Bores 80, 72 have a circular cross-section and are sized to permit a clearance and sliding fit, respectively, with the guide pin during installation of the nail body 62 into the intramedullary canal. After insertion of the guide pin is completed, an insertion/removal instrument (not shown) is attached to the proximal end of the nail assembly. The instrument utilizes internal threads 82 for attachment. The internal threads 82 (FIG. 6a) at the proximal end of the nail body 62 provide for instrument interface and sleeve lock screw 67 insertion. The instrument includes a protruding feature which mates simultaneously with slot 69 of nail body 62. Slot 69 provides angular alignment between the instrument and nail body 62. One of ordinary skill in the art would appreciate that from the description of the various embodiments of the invention provided herein one or more suitable insertion/removal instruments can be derived, making a description of an insertion/removal instrument unnecessary.

After the instrument is attached, nail body 62 is inserted into the femur and the guide pin (not shown) is removed. Lag screw 66 is then inserted through nail body slot 74. A guide pin may also be used to ensure proper placement of lag screw 66. The external features of the lag screw 66 are indicated in FIG. 7b and include the threads 102, body 104 and the flats 106 on the body 104. The threads 102 engage the cancellous bone within the femoral head on the proximal side of the fracture line.

Instrumentation assures proper insertion depth of lag screw 66 and alignment of the plane of lag screw flats 106 parallel to the nail body proximal bore 80 longitudinal axis. After the lag screw 66 is implanted in its proper position within the femur, its trailing end protrudes partially or fully through slot 74 of nail body 62.

Washer 64 is then placed alongside the bone in a manner that allows sleeve 110 to be inserted through washer hole 86 (FIG. 7c) and into slot 74 in order to secure screw 66.

FIG. 7c illustrates a washer according to an embodiment of the present invention. Washer 64 includes screw holes 87 and sleeve hole 86. While washer 64 includes 4 screw holes 87, in an alternative embodiment, a washer has fewer or greater holes, or even no holes. FIG. 7d illustrates a washer according to an alternative embodiment of the present invention. Washer 84 includes screw holes 88 and sleeve hole 89. One of ordinary skill in the art would appreciate that in an alternative embodiment, a washer is not used with nail 60, and lip 83 of sleeve 110 pushes against the femoral bone directly preventing axial translation of sleeve 110. A washer may be needed if there is substantial comminution at the surface of the bone through which sleeve 110 enters the femur.

Sleeve 110 is utilized to secure lag screw 66 in slot 74 after implantation of lag screw 66 in the femur. The distance F between the flat surfaces 112 (FIG. 12d) is sized for a sliding fit in slot 74. Sleeve 110 has a circular bore 96 and a small length of bore having oppositely disposed flats 111 at the leading end (FIGS. 12a, 12c, 12d). These are sized for a sliding fit with the body and flats 106 of lag screw 66 thus allowing axial translation of lag screw 66 but not allowing relative rotation. Flat surfaces 112 engage the walls of slot 74 to prevent relative rotation between sleeve 110 and slot 74. Counterbore 114 is provided in the end of sleeve 110 opposite that of the flats 111 and has the configuration as shown in FIGS. 12c, 12e. It is sized and configured for mating with compression screw 65 as shown in FIG. 7a.

To secure lag screw 66, the leading end of sleeve 110 containing flats 111 is inserted into slot 74 and the bore 96 of sleeve 110 aligned, with the aid of instrumentation (not shown) with the similarly shaped lag screw body 104. The sleeve 110 is inserted further into slot 74 thus mating with lag screw 66. Since, as described previously, sleeve flats 111 interact with lag screw flats 106 preventing relative rotation between lag screw 66 and sleeve 110 and the plane of lag screw flats 106 are already aligned parallel to nail proximal bore 80 longitudinal axis the plane of the sleeve flats 111 are now also aligned parallel with the nail proximal bore 80 longitudinal axis.

FIG. 11a is an isometric view of a sleeve lock according to an embodiment of the present invention. FIG. 11b is a front view of FIG. 11a. The nail body 62 insertion instrument (not shown) is then removed from the proximal end of nail body 62 and the locking rod 108 (FIGS. 11a, 11b) end of sleeve lock screw 67 is inserted into the proximal bore 80 of the nail body 62. Sleeve lock screw 67 (FIGS. 11a, 11b), has a basic cylindrical cross section. The outside diameter of sleeve lock screw 67 is sized such that it passes through internal threads 82 and fits in proximal bore 80 of nail body 62. Threads 100 of sleeve lock screw 67 are screwed into threads 82 until rod 108 makes contact with upper surface 79 of sleeve 110 and screw 67. Depending upon the starting location of lower surface 75 of slot 74 when screw 67 is screwed into threads 82, lower surface 75 of slot 74 will rise while screw 67 is being screwed into bore 80 until it makes contact with lower surface 77 of sleeve 110. Screw 67 is screwed until it cannot be screwed into bore 80 any further. If sleeve lock screw 67 cannot be screwed any further, lower surface 75 of slot 74 will have made contact with lower surface 77 of sleeve 110. If sleeve lock screw 67 cannot be screwed any further after rod 108 makes contact with the upper surface of sleeve 110, sleeve 110 is fixed in rotation, axial translation, and longitudinal translation. The sleeve lock screw 67, sleeve 110 and lag screw 66 are now in the relative positions as shown in FIG. 7a. Similarly, in the case of sleeve 63 of FIG. 6d, sleeve 63 and lag screw 66 are now in the relative positions as shown in FIG. 6e.

One of ordinary skill in the art would appreciate that the insertion instrument (not shown) may also be used to raise lower surface 75 of slot 74 so that it makes contact with lower surface 77 of sleeve 110. When the insertion instrument is attached to nail body 62 via internal threads 82, nail body 62 can be raised by pulling on the insertion instrument with a movement that is opposite of that used to insert nail body 62 into the intramedullary canal. When the insertion instrument is used to raise lower surface 75 of slot 74 so that it makes contact with lower surface 77 of sleeve 110, sleeve lock screw 67 does not have to be used to raise lower surface 75.

With sleeve 110, sleeve lock screw 67 and lag screw 66 assembled as shown in FIG. 7a within nail body 62, sleeve 110 is fixed in rotation by interaction of sleeve flats 112 with slot 74 and in longitudinal translation by interaction of lip 83 of sleeve 110 with washer 84 which is attached with washer screws 85 to the femur. Since sleeve 110 is now fixed in rotation, lag screw 66 is also fixed in rotation by the interaction of sleeve flats 111 and lag screw flats 106 but not fixed in translation. One of ordinary skill in the art would appreciate that an alternative embodiment would not include washer screws 85 and washer 64 is not attached to the femur with screws but rather is pushed against the femur due to the compressive force of created by the combination of compression screw 65 and lip 83 of sleeve 110 as described elsewhere herein.

FIG. 12e is a front view of the sleeve of FIG. 12a according to an alternative embodiment of the present invention. With sleeve 110 and lag screw 66 fixed in rotation, compression screw 65 is inserted through bore 96 of sleeve 110 mating its threaded end with internal threads within lag screw 66 and its head with sleeve counterbore 114 (FIGS. 12c, 12e). As compression screw 65 is tightened, its head contacts sleeve counterbore 114, and since sleeve 110 is fixed in longitudinal translation by lip 83 and washer 84, lag screw 66 is drawn toward nail body 62 thereby compressing the fracture in the femoral neck and head.

One or two or more cortical screws 68 can now be used to fix nail body 62 both in translation and rotation within the intramedullary canal. The cortical screws 68 are placed through the lateral femoral cortex and through clearance holes 78 in the nail body 62, then through the medial femoral cortex (FIG. 7a). The clearance holes 78 of nail body 62 pass through the distal outside surface and wall of the nail body 62, into the distal bore 72 and continue on the same axis through the opposite wall and outer diameter. Their diameter is such as to allow passage of the threaded portion of the cortical screw 68. The nail body 62 is secured both in axial translation and rotation within the intramedullary canal by cortical screws 68 when they are installed through the lateral cortex, clearance holes 78, and the medial cortex of the femur as illustrated in FIG. 7a.

One of ordinary skill in the art would appreciate that, in an alternative embodiment, one or more of the clearance holes, holes in lock plates, or holes in washers are threaded ones which can allow a compressive force to be applied laterally between the lateral femoral cortex and the nail body. Hole 207 of FIG. 5e is a threaded hole with inner threads 208, but in an alternative embodiment any of the holes described herein are threaded holes. Screw 217 of FIG. 5a includes metal engaging threads 219 that engage threads 208 of hole 207 and bone engaging threads 218. In an alternative embodiment, metal engaging threads 219 of screw 217 engage the complementary inner threads 208 of hole 207. In an alternative embodiment, screw 217 includes only metal engaging threads 219. In an alternative embodiment one or more screws 68 includes metal engaging threads, bone engaging threads, or both.

The nail assembly can be removed by reversing the assembly order. The nail assembly can be removed by removing cortical screws 68, compression screw 65, washer screws 85, removing sleeve lock screw 67, releasing sleeve 110, removing washer 64 and lag screw 66 and utilizing nail body internal threads 82 to interface a nail body 62 removal instrument (not described) and pull the nail body from the intramedullary canal.

FIG. 12a is an isometric view of a sleeve according to an alternative embodiment of the present invention. Sleeve 110 is largely tubular in shape. However, unlike sleeve 63 which is a combination of a tubular shape and a plate which the tubular shape intersects at 90°, sleeve 110 is a combination of a tubular shape and plate 124 which the tubular shape intersects at an angle greater than 90°. In both cases, for sleeve 110 and sleeve 63, the region of intersection is hollow. FIG. 12b illustrates a side view of the sleeve of FIG. 12a according to an alternative embodiment of the present invention. Angle P (FIG. 12b) between the bore axis of sleeve 110 and the plane of plate 113 is 135°. However, in an alternative embodiment the angle between the sleeve's bore axis and an intersecting plate may be larger or smaller than 135°. A sleeve with angle P equal to 135° can be used in cases where the lag screw is inserted at an angle between 130° and 140° and in cases outside that range.

FIG. 13a is a side view of a sleeve according to yet another alternative embodiment. Angle P′ between the bore axis of sleeve 160 and the plane of plate 161 is 130°. A sleeve with angle P′ equal to 130° can be used in cases where the lag screw is inserted at angle between 125° and 135° and in cases outside that range. FIG. 13b is a side view of a sleeve according to yet another alternative embodiment. Angle P″ between the bore axis of sleeve 170 and the plane of plate 171 is 140°. A sleeve with angle P″ equal to 140° can be used in cases where the lag screw is inserted at an angle between 135° and 145° and in cases outside that range. The cross sectional views, front views, and isometric views of sleeves 160, 170 are similar to the same views of sleeve 110 and need not be illustrated herein.

One of ordinary skill in the art would appreciate that the length D (FIG. 12c) of the upper surface of sleeve 110 is an implementation consideration that need not be specified herein and can vary depending upon the embodiment. Length D′ (FIG. 13a) of sleeve 160 of an alternative embodiment of the present invention is less than length D of sleeve 110 of yet another alternative embodiment of the present invention. One of ordinary skill in the art would appreciate that length S (FIG. 5a) of screw 66 is an implementation consideration and can vary depending upon the embodiment.

Sleeve 63 includes inner flats 301 and outer flats 302 which perform a function similar to that performed by flats 111, 112 respectively. One of ordinary skill in the art would appreciate that in an alternative embodiment, there is only one flat 111 on the inner surface of sleeve 110. One of ordinary skill in the art would appreciate that in an alternative embodiment, there is only one flat 112 on the outer surface of sleeve 110. Furthermore, one of ordinary skill in the art would appreciate that in an alternative embodiment, there is only one flat 111 on the inner surface of sleeve 110 and one flat 112 on the outer surface of sleeve 110.

FIG. 12c is a cross section view of the sleeve of FIG. 12a. The sleeve axial bore axis is indicated by the dashed line in FIG. 12c. FIG. 12d is a view from the front of the sleeve of FIG. 12a and looking down the sleeve bore. Body 104 of lag screw 66 has two flats 106 180° apart (FIG. 7b) which interface with bore 96 and flats 111 (FIGS. 12a, 12c, 12d) of sleeve 110 in such a way as to allow axial translation or slide of the lag screw while preventing rotation relative to sleeve 110. This sliding prevents penetration of the femoral head by the proximal end of the lag screw as the fracture compresses from patient load bearing.

FIG. 12f is a front view of a sleeve according to an alternative embodiment of the present invention. Sleeve 180 has an integrated washer 182 that includes holes 184 for screws for attaching sleeve 180 to the femoral bone. FIG. 12g is an isometric view of the sleeve of FIG. 12f. FIG. 12h is a cross section view of the sleeve of FIGS. 12f, 12g.

FIG. 8 is an isometric view of a sleeve according to an alternative embodiment of the present invention. FIG. 9 is a top of view of FIG. 8. FIG. 10 is a cross section view of FIG. 8. The sleeve axial bore axis is indicated by the dashed line in FIG. 10. Body 104 of lag screw 66 has two flats 106 180° apart (FIG. 7b) which interface with bore 405 and flats 402 (FIGS. 8, 9, 10) of sleeve 400 in such a way as to allow axial translation or slide of the lag screw while preventing rotation relative to sleeve 110. This sliding prevents penetration of the femoral head by the proximal end of the lag screw as the fracture compresses from patient load bearing. Flat surfaces 404 engage the walls of slot 74 to prevent relative rotation between sleeve 400 and slot 74.

In an alternative embodiment, sleeve 400 is utilized to secure lag screw 66 into slot 74 after implantation of lag screw 66 in the femur. The distance G between the flat surfaces 402 (FIG. 9) is sized for a sliding fit in slot 74. Sleeve 400 has a circular bore 405 and a small length of bore having oppositely disposed flats 401 at the leading end (FIGS. 8, 9, 10). These are sized for a sliding fit with the body and flats 106 of lag screw 66 thus allowing axial translation of lag screw 66 but not allowing relative rotation. Counterbore 406 is provided in the end of sleeve 400 opposite that of the flats 401 and has the configuration as shown in FIG. 10. It is sized and configured for mating with compression screw 65 in a manner similar to counterbore 114 of sleeve 110 as shown in FIG. 7a. Sleeve 400 includes angled lips 401.

FIG. 8a is an isometric view of a sleeve according to an alternative embodiment of the present invention. FIG. 9a is a top of view of FIG. 8a. FIG. 10a is a cross section view of FIG. 8a. The sleeve axial bore axis is indicated by the dashed line in FIG. 10a. Body 104 of lag screw 66 has two flats 106 180° apart (FIG. 7b) which interface with bore 305 and flats 301 (FIGS. 8, 9, 10) of sleeve 63 in such a way as to allow axial translation or slide of lag screw 66 while preventing rotation relative to sleeve 110. Rotation of sleeve 110 relative to slot 74 is prevented by the interface of flats 302 with the walls of slot 74. This sliding prevents penetration of the femoral head by the proximal end of the lag screw as the fracture compresses from patient load bearing.

In an alternative embodiment, sleeve 63 is utilized to secure lag screw 66 into slot 74 after implantation of lag screw 66 in the femur. The distance Q between the flat surfaces 302 (FIG. 9a) is sized for a sliding fit in slot 74. Sleeve 110 has a circular bore 305 and a small length of bore having oppositely disposed flats 301 at the leading end (FIGS. 8a, 9a, 10a). These are sized for a sliding fit with the body and flats 106 of lag screw 66 thus allowing axial translation of lag screw 66 but not allowing relative rotation. Counterbore 306 is provided in the end of sleeve 63 opposite that of the flats 301 and has the configuration as shown in FIG. 10a. It is sized and configured for mating with compression screw 65 in a manner similar to counterbore 114 of sleeve 110 as shown in FIG. 7a.

FIG. 12i is a cross section view of a sleeve according to an alternative embodiment of the present invention. Sleeve 310 is similar in an isometric view to sleeve 110. The salient feature that differentiates sleeve 310 from sleeve 110 are inner threads 315. Sleeve 310 includes inner threads 315 that engage complementary threads of an end cap that limits the longitudinal translation of a compression screw such as compression screw 65. FIG. 12j is another cross section view of the sleeve FIG. 12i with a compression screw and an end cap restraining the compression screw. End cap 116 when disposed within sleeve 310 limits the longitudinal movement of compression screw 65. FIG. 12k is an isometric view of an end cap for a sleeve according to an embodiment of the present invention. End cap 116 includes outer threads 118 that engage inner threads 315 of sleeve 310. FIG. 12i is a front view of the end cap of FIG. 12k. In an alternative embodiment, sleeve 110, or any of the other sleeves described herein, has inner threads such as inner threads 315 of sleeve 310.

FIG. 5c is a longitudinal view of a nail body according to an alternative embodiment of the present invention. FIG. 5d is a front view of the nail body of FIG. 5c according to an alternative embodiment of the present invention. FIG. 5e is a view, in a longitudinal cross section, of the nail body of FIG. 5c according to an alternative embodiment of the present invention. Nail body 200 of FIGS. 5c, 5d, 5e has slot 201 with slot height H′, where H′<H. Slot height H is the height of slot 74 of nail body 62. One of ordinary skill in the art would appreciate that the present invention is not limited to any particular value or values for slot height so long as the slot height permits a range of angles of insertion for a sleeve and screw. Body 200 includes a clearance slot 204 which is sized to allow a screw to be positioned at various positions within slot 204. One of ordinary skill would appreciate that any of the clearance holes described herein can be clearance slots in alternative embodiments of the invention.

FIG. 5f is a longitudinal view of a nail body according to an alternative embodiment of the present invention. FIG. 5g is a front view of the nail body of FIG. 5f according to an alternative embodiment of the present invention. FIG. 5h is a view, in a longitudinal cross section, of the nail body of FIG. 5f according to an alternative embodiment of the present invention. Nail body 210 of FIGS. 5f, 5g, 5h has slot 211 which intersects proximal bore 212 at an angle N other than 90°. As a consequence, slot 211 has an opening on the proximal side of body 210 and an opening on the distal side of body 210 that are staggered, or longitudinally (and/or radially) offset with respect to each other, rather than aligned. One of ordinary skill in the art would appreciate that the present invention is not limited to any particular value or values for angle N so long as the angle N and slot height in relation to the dimensions of the sleeve and screw permit a range of angles of insertion for a sleeve and screw.

FIG. 14 is a view, longitudinal cross section, of an intramedullary nail system according to an alternative embodiment of the present invention placed in the intramedullary canal of a bone in a retrograde fashion with a lock plate disposed medially. Nail 150 includes slot 152 which allows lag screw 154 and sleeve 156 to be inserted into slot 152 in order to treat fractures of the distal femur. Lock plate 158 includes an opening 160 that largely corresponds to the dimensions of slot 152 in that it is shaped in a manner that allows screw 154 to be inserted into slot 152 at the range of angles supported by slot 152.

FIG. 14a is a front view of the lock plate in FIG. 14 according to an alternative embodiment of the present invention. Depending upon the embodiment, lock plate 158 and other lock plates or washers described herein are made so as to have a shape that anatomically matches the general shape of the type bone or area of bone to which they are to abut against. In an alternative embodiment, a lock plate 158 or other plate described herein which is to be disposed medially would have a different shape than one which is to be disposed laterally.

FIG. 14b is a front view of the lock plate in FIG. 14 and the sleeve of FIG. 14 as placed in the opening of the washer according to an alternative embodiment of the present invention. FIG. 14c is a side view of the lock plate in FIGS. 16, 16a, 16b. In an alternative embodiment a lock plate is not used. Opening 160 of lock plate 158 has a width W′ at a portion of the proximal end that is wider than the width W″ at a distal end of opening 160 and the width W′″ of plate 159 of sleeve 156, thereby allowing opening 160 to pass plate 159. Lock plate 158 is then slid into the proper position along side the bone which results in plate 159 overlapping lock plate 158. Compression screw 153 is then screwed into the distal end of screw 154 resulting in a compressive force being applied across a fracture in the base of the femur. Lock plate 158 includes screw holes 162 for receiving screws (not shown) that attach washer 158 to the bone at the base of the femur.

Sleeve lock screw 67 (not shown in FIG. 14) is used in an embodiment to lock sleeve 156 as described in connection with FIG. 7a. However, unlike the situation described in FIG. 7a, the surface at the leading end of slot 152 need not be raised by an insertion instrument or by screwing in screw 67 through the trailing axial bore of nail 150.

FIG. 15 is a view, in longitudinal cross section, of an intramedullary nail system according to an alternative embodiment of the present invention placed in the intramedullary canal of a bone in a retrograde fashion with a lock plate disposed laterally.

Nail 220 includes slot 222 which allows lag screw 224 and sleeve 226 to be inserted into slot 222 in order to treat fractures at the distal femur. Lock plate 228 includes an opening 260 that largely corresponds to the dimensions of slot 222 in that it is shaped in a manner that allows screw 224 to be inserted into slot 222 at the range of angles supported by slot 222. FIG. 15a is a front view of the lock plate in FIG. 15 according to an alternative embodiment of the present invention. FIG. 15b is a side view of the lock plate in FIGS. 15, 15a. In an alternative embodiment, a lock plate is not used. Opening 260 of lock plate 228 passes the plate (not shown) of sleeve 226. Lock plate 228 is then slid into the proper position along side the bone which results in the plate of sleeve 226 overlapping lock plate 228. Compression screw 223 is then screwed into the distal end of screw 224 resulting in a compressive force being applied across a fracture in the distal femur. Lock plate 228 includes screw holes 262 for receiving screws (not shown) that attach lock plate 228 to the distal femur.

Sleeve lock screw 67 (not shown in FIG. 15) is used in an embodiment to lock sleeve 226 as described in connection with FIG. 7a. However, unlike the situation described in FIG. 7a, the surface at the leading end of slot 222 need not be raised by an insertion instrument or by screwing in screw 67 through the trailing axial bore of nail 220.

In an alternative embodiment, the sleeve lock screw includes an end cap (not shown) and a sleeve lock rod (not shown) that is separate from the end cap. The end cap includes outer threads that are adapted to engage complementary inner threads 82 of nail body 62. The end cap is adapted to press against a trailing end of the sleeve lock rod when the sleeve lock rod is disposed in the proximal bore of the trailing end of nail body 62 so as to push the sleeve lock rod against the upper surface of the sleeve.

The steps for inserting nail 220 into the distal femur are similar to those described for inserting nail body 62 in an antegrade fashion into the femur. The previous description for inserting nail body 62 into the femur is incorporated herein by reference and will not be repeated. One of ordinary skill in the art would appreciate any modifications that would be necessary to be made to the previous description.

As described herein, a femoral universal nail (FUN) in accordance with an embodiment of the present invention allows sliding and compression of the proximal fracture site and can do so dynamically after an operation. A FUN in accordance with an embodiment of the present invention allows placement of guide pins and subsequent screws in ideal positions and with flexibility. The slot of a FUN in accordance with an embodiment of the present invention allows insertion of a second or third screw, as needed. In an embodiment, a polyaxial sleeve and plate combination allows compression as well as sliding if needed. In an embodiment, a slotted side plate can be used to address comminution of the greater trochanter.

In an embodiment, the shaped surfaces of the shaft of a lag screw and the sleeve lock will control relative rotation while still allowing for compression and sliding. Inserting the lag screw in a slot rather than a fixed hole, will allow for adaptation and placement of the proximal screw in the most stable bone and ideal positioning. Additionally, it will avoid the valgus and varus mal-reduction and fixation.

A FUN in accordance with an embodiment of the present invention allows fixation of all proximal femoral fractures, shaft fractures and any combinations of the foregoing. A FUN in accordance with an embodiment of the present invention is used as a retrograde IM rod. At the same time, a FUN in accordance with an embodiment of the present invention addresses the complex distal femur fracture with the flexibility of its polyaxial slotted side plate and allows the insertion of multiple screws, locked and unlocked, while avoiding stripping of soft tissues and devitalizing bone fragments. A FUN in accordance with an embodiment of the present invention is more mechanically sound since the fixation is IM and because it provides for weight sharing.

A femoral universal nail addresses many kinds of femur fracture. It is versatile and easy to use while combining sound biomechanical principles and avoiding the deficiencies inherent in other devices.

In the preceding specification, the invention has been described with reference to specific exemplary embodiments of the invention. It will, however, be evident to one of ordinary skill in the art that various modifications and changes may be made without departing from the broader spirit and scope of the invention as set forth in the claims that follow. The specification and drawings are accordingly to be regarded in an illustrative rather than restrictive sense.

1. An intramedullary nail for insertion in the intramedullary canal of a long bone, the nail comprising:
a nail body having a leading end, and a trailing end,
the trailing end having an axial bore, axial bore axis, and an intersecting transverse slot;
a sleeve for a lag screw for purchase in bone, wherein the transverse slot is adapted to receive the sleeve, the sleeve has an axial bore and an axial bore axis, the transverse slot has a first opening and a second opening both adapted to allow the sleeve to be movably disposed within the transverse slot and provide alternative angles for the sleeve axial bore axis with respect to the axial bore axis of the trailing end; and
a sleeve lock, wherein the axial bore is adapted to accept the sleeve lock, and the sleeve lock has a leading end that is to contact an upper surface of the sleeve to prevent axial movement of the sleeve within the transverse slot and away from the leading end of the nail body.

2. The nail of claim 1, wherein the sleeve has an angulated trailing end with a lip that is adapted to press against bone so as to limit longitudinal movement of the sleeve through the transverse slot.

3. The nail of claim 2, further comprising a washer, and wherein the washer is disposed between the lip and the bone.

4. The nail of claim 1, wherein the transverse slot is a transverse bore with a bore diameter that is larger than an outer diameter of the sleeve so as to provide alternative angles for the sleeve axial bore axis with respect to the axial bore axis of the trailing end.

5. The nail of claim 1, further comprising a lag screw having external threads for purchase in bone, wherein the lag screw has a leading end and a trailing end, the sleeve has a trailing end and a leading end that is approximately co-terminus with the trailing end of the lag screw, and the co-terminus ends are adapted for longitudinal translation relative to each other to transmit compressive force between the trailing end of the sleeve and the leading end of the lag screw.

6. The nail of claim 5, further comprising a compression screw having a leading end with external threads and a trailing end that is to act against the trailing end of the sleeve to limit insertion of the compression screw into the sleeve, the trailing end of the lag screw has internal threads that are complementary to the external threads of the compression screw, and the external threads are adapted to engage the internal threads to produce longitudinal translation relative to each other.

7. The nail of claim 5, wherein the sleeve has a shaped inner surface, the trailing end of the lag screw has a shaped outer surface, and the shaped inner surface and the shaped outer surface prevent relative rotation between the lag screw and the sleeve.

8. The nail of claim 5, wherein the sleeve has a shaped outer surface to prevent the sleeve from having relative rotation with the transverse slot.

9. The nail of claim 1, further comprising a lock plate that includes a lock plate slot adapted to pass therethrough the sleeve and provide alternative angles for the sleeve axial bore axis with respect to the axial bore axis of the trailing end.

10. The nail of claim I, wherein the nail body has at least one clearance hole that includes inner threads.

11. The nail of claim 10, further comprising a cortical screw with outer threads for engaging the inner threads.

12. The nail of claim 11, wherein the cortical screw includes bone engaging threads dimensioned to pass through the at least one clearance hole.

13. The nail of claim 1, further comprising an end cap, the end cap having outer threads, the sleeve has a trailing end with inner threads that are complementary to the outer threads of the end cap permitting the end cap to be screwed into the trailing end of the sleeve in order to limit longitudinal movement of a compression screw that is coupled to the lag screw.

14. The nail of claim 1, wherein the transverse slot includes an upper region and a lower region and both the upper region and lower region remain hollow after the sleeve is passed therethrough thereby allowing the nail to be moved axially to raise the leading end of the nail body towards the sleeve.

15. A lag screw stabilizer for insertion into a transverse slot of a nail body for insertion in the intramedullary canal of a bone, the lag screw stabilizer comprising:
a sleeve for a lag screw, the sleeve having a sleeve axis and an angulated trailing end, the nail body having a trailing end and a proximal diametral axis, the trailing end having a transverse slot adapted to receive the sleeve therethrough and to provide alternative angles for the sleeve axis with respect to the proximal diametral axis, the sleeve having a shaped outer surface that is adapted to engage the transverse slot so as to prevent relative rotation between the sleeve and the transverse slot, and the sleeve having a shaped inner surface that is to prevent relative rotation between the sleeve and a lag screw.

16. The lag screw stabilizer of claim 15, further comprising a washer that has a hole for passing the sleeve, wherein the washer is adapted to be disposed between a lip on the angulated trailing end of the sleeve and bone.

17. The lag screw stabilizer of claim 16, wherein the angulated trailing end includes an integrated washer.

18. An intramedullary nail for insertion in the intramedullary canal of a bone, the nail comprising:
a nail body having a leading end, a trailing end, and a proximal diametral axis,
the trailing end having an axial bore and an intersecting transverse slot,
the transverse slot adapted to pass a sleeve for a lag screw, the sleeve having a sleeve bore axis, and the transverse slot adapted to provide alternative angles for the sleeve bore axis with respect to the proximal diametral axis; and
a sleeve lock screw, wherein the axial bore includes threads that are adapted to engage complementary threads of the sleeve lock screw, the axial bore adapted to pass therethrough the sleeve lock screw to make contact with an upper surface of the sleeve when the sleeve is disposed within the transverse slot, and a lower surface of the transverse slot and the sleeve lock screw restrict axial movement of the sleeve within the transverse slot and away from the leading end.

19. The nail of claim 18, further comprising a lock plate that includes a lock plate slot adapted to pass therethrough the sleeve and provide alternative angles for the sleeve axial bore axis with respect to the axial bore axis of the trailing end.

20. The nail of claim 18, wherein the transverse slot is entirely hollow when the sleeve is passed therethrough.

21. An intramedullary nail for insertion in the intramedullary canal of a long bone, the nail comprising:
a nail body having a leading end, and a trailing end,
the trailing end having an axial bore, and axial bore axis, and an intersecting transverse slot,
the transverse slot having two parallel walls and adapted to receive a sleeve for a screw, the sleeve has an axial bore and an axial bore axis, wherein the transverse slot is adapted to allow the sleeve to be movably disposed within the transverse slot so as to have the sleeve axial bore axis be at one of several angles with respect to the axial bore axis of the trailing end, and
wherein the axial bore is adapted to accept a sleeve lock that has a leading end that is to contact an upper surface of the sleeve to prevent axial movement of the sleeve within the transverse slot and away from the leading end of the nail body.

22. The nail of claim 21, further comprising:
the sleeve;
the screw, wherein the screw is a lag screw; and
wherein the lag screw has a leading end and a trailing end, the sleeve has a leading end that is approximately co-terminus with the trailing end of the lag screw, and the co-terminus ends are adapted for longitudinal translation relative to each other to transmit compressive force between the trailing end of the sleeve and the leading end of the lag screw.

23. The nail of claim 22, further comprising a compression screw having a leading edge with external threads, the compression screw is adapted to be inserted into the sleeve, the trailing end of the lag screw has internal threads that are complementary to the external threads, and the external threads are adapted to engage the internal threads to produce longitudinal translation relative to each other.

24. The nail of claim 22, wherein the sleeve has a shaped outer surface that is to prevent relative rotation between the sleeve and the transverse slot.

25. The nail of claim 22, wherein the sleeve has a shaped inner surface, the trailing end of the lag screw has a shaped outer surface, and the shaped inner surface and the shaped outer surface prevent relative rotation between the lag screw and the sleeve.

26. The nail of claim 22, wherein the trailing end of the sleeve is angulated.

27. The nail of claim 21, further comprising the sleeve lock.

28. An intramedullary nail for insertion in the intramedullary canal of a long bone, the nail comprising:
a nail body having a leading end, a trailing end, and a proximal diametral axis,
the trailing end having an axial bore and an intersecting transverse slot,
the transverse slot adapted to receive a sleeve for a lag screw, the sleeve having a sleeve axis, and the sleeve after insertion through the transverse slot is capable of being disposed in the transverse slot so as to have the sleeve axis be at one of several angles with respect to the diametral axis, and
the axial bore adapted to accept a sleeve lock screw that has a leading end that is to contact an upper surface of the sleeve to prevent axial movement of the sleeve within the transverse slot and away from the leading end of the nail body.

29. The nail of claim 28, further comprising:
the sleeve; and
a washer, wherein the sleeve has a shaped trailing edge that is to engage the washer that is adapted to press against bone so as to prevent longitudinal movement of the sleeve within the transverse slot.

30. The nail of claim 28, further comprising the sleeve lock screw.

31. The nail of claim 28, further comprising the sleeve, the sleeve having a shaped surface adapted to prevent the sleeve from having relative rotation with the transverse slot.

32. A lag screw stabilizer for insertion into a transverse slot of an intramedullary nail, the lag screw stabilizer comprising:
a sleeve for a lag screw, the sleeve having a sleeve axis, the intramedullary nail having a trailing end, and a proximal diametral axis, the trailing end having a transverse slot adapted to receive the sleeve and to permit the sleeve to be disposed in the transverse slot so as to have the sleeve axis be at one of several angles with respect to the proximal diametral axis, the sleeve having a shaped outer surface that is adapted to prevent relative rotation between the sleeve and the transverse slot, the sleeve having a shaped inner surface that is to prevent relative rotation between the sleeve and the lag screw, the lag screw has a leading end and an angulated trailing end, the sleeve has a leading end that is approximately co-terminus with the trailing end of the lag screw, and the co-terminus ends are adapted for longitudinal translation relative to each other to transmit compressive force between the trailing end of the sleeve and the leading end of the lag screw, the angulated trailing end of the sleeve has a lip that is adapted to press against bone so as to limit longitudinal movement of the sleeve through the transverse slot, the trailing end of the intramedullary nail has an axial bore that is adapted to accept a sleeve lock screw, and wherein the sleeve lock screw has a leading end that is to contact an upper surface of the sleeve to prevent axial movement of the sleeve within the transverse slot and away from the leading end of the intramedullary nail.

33. An intramedullary nail for insertion in the intramedullary canal of a long bone, the nail comprising:
a nail body having a leading end, a trailing end, and a proximal diametral axis,
the trailing end having an axial bore and an intersecting transverse bore,
the transverse bore having a transverse bore diameter and adapted to receive a sleeve for a lag screw and to permit the sleeve to be slidably and axially disposed in the transverse slot at one of several angles with respect to the diametral axis,
the sleeve having an angulated trailing end and an outer diameter that is less than the transverse bore diameter, and the axial bore adapted to accept a sleeve lock screw, the sleeve lock screw has a leading end that is to contact an upper surface of the sleeve to prevent axial movement of the sleeve within the transverse bore and away from the leading end of the nail body.

34. The nail of claim 33, further comprising the sleeve lock screw.

35. An intramedullary nail for insertion in the intramedullary canal of a long bone, the nail comprising:
a nail body having a leading end, and a trailing end,
the trailing end having an axial bore, and axial bore axis, and an intersecting transverse slot,
the transverse slot having two parallel walls and adapted to receive a sleeve for a lag screw, the sleeve has an axial bore and an axial bore axis, wherein the transverse slot is adapted to allow the sleeve to be movably disposed within the transverse slot so as to have the sleeve axial bore axis be at one of several angles with respect to the axial bore axis of the trailing end,
the axial bore is adapted to accept a sleeve lock that has a leading end that is to contact an upper surface of the sleeve to prevent axial movement of the sleeve within the transverse slot and away from the leading end of the nail body,
the lag screw has a leading end and a trailing end, the sleeve has a leading end that is approximately co-terminus with the trailing end of the lag screw, the co-terminus ends are adapted for longitudinal translation relative to each other to transmit compressive force between the trailing end of the sleeve and the leading end of the lag screw,
the sleeve has a shaped outer surface that is to prevent relative rotation between the sleeve and the transverse slot,
the sleeve has a shaped inner surface, the trailing end of the lag screw has a shaped outer surface, and the shaped inner surface and the shaped outer surface prevent relative rotation between the lag screw and the sleeve, and
wherein the trailing end of the sleeve is angulated and is adapted to receive a compression screw having a leading edge with external threads, the trailing end of the lag screw has internal threads that are complementary to the external threads of the compression screw, and the external threads of the compression screw are adapted to engage the internal threads of the lag screw to produce longitudinal translation relative to each other.
